(19) United States
(12) Patent Application Publication
(10) Pub. No.: US 2024/0397938 A1
(43) Pub. Date: Dec. 5, 2024

(54) DEVICE AND METHOD FOR MONITORING A TRANSPLANT

(71) Applicant: GRAFT CENTRAL, Montpellier (FR)

(72) Inventor: Clement LABICHE, Montpellier (FR)

(21) Appl. No.: 18/687,840

(22) PCT Filed: Aug. 30, 2022
(86) PCT No.: PCT/EP2022/074134

§ 371 Date: Feb. 29, 2024

(30) Foreign Application Priority Data
Aug. 30, 2021 (FR) FR2109045

(51) Int. Cl. A01N 1/02; G16H 40/40

(52) U.S. Cl. CPC A01N 1/0273; G16H 40/40

(57) ABSTRACT

The device (10) for monitoring a graft (12) comprises, in a standalone unit (11) configured to accompany the graft in a graft transport case (14), a graft temperature sensor and a means for remotely communicating the temperature of the graft.

### TECHNICAL FIELD OF THE INVENTION

The present invention relates to a device and method for graft monitoring. It applies, in particular, to the monitoring of grafts during their transport from a hospital where a donor is located to a hospital where a recipient is located.

### STATE OF THE ART

It is noted that the term “transplant” covers both organ or tissue transplants and blood transfusions, these transplanted or transfused elements being called “grafts”.

During the transport of a graft, which can take many hours, many environmental constraints must be complied with continuously so that the graft is not damaged and to minimise the risks of rejection by the recipient's body. Containers are known, such as those described in utility certificate CN202255520U, which monitor changes in the temperature. Utilisation of these containers requires the replacement of all the transport equipment, and their functions are too limited to ensure the quality of the graft transport.

Patent application US 2011/173 023 discloses an organ transport device comprising a temperature sensor. Patent application WO 2020/192 513 discloses a device for organ preservation with mechanical perfusion. Patent application AU 2019 213 360 discloses a transportable device for the preservation and monitoring of a lung outside of the body. These devices are especially costly and unsuitable for graft transports in countries with a small medical budget.

### DESCRIPTION OF THE INVENTION

The present invention aims to remedy all or part of these drawbacks.

To this end, the present invention envisions a graft monitoring device which comprises, in a standalone unit configured to accompany the graft in a graft transport case, a graft temperature sensor and a means for remotely communicating the temperature of the graft.

Thanks to these provisions, any graft transport case can be transformed into a communicating case. In addition, the unit is physically associated with the graft since it can, in the environment of the interior of the transport case, monitor all the variations in transport conditions and, in particular, changes in the temperature of the graft. These transport conditions are regularly sent to the recovery and transplant coordinators, the surgeons, the members of the transport team in charge of escorting the graft and/or the members of the medical team waiting for the graft to transplant it. The quality of the transport can therefore be evaluated and the change in the graft's ability to be transplanted can be estimated.

In some embodiments, the device also comprises sensors capturing values of physiological parameters of the graft.

In some embodiments, the device also comprises a means for making predictions about changes in the values of physiological parameters while the graft is being transported.

In some embodiments, the prediction means is based on machine learning performed using physiological parameter values obtained during previous transports.

In some embodiments, the prediction means is configured to make at least one prediction of physiological parameter values for the time when the graft arrives at the site of its transplant into the body of a recipient patient.

In some embodiments, the device comprises a means for geolocation and evaluation at the time when the graft arrives at the site of its transplant into the body of a recipient patient.

In some embodiments, the prediction means is configured to propose at least one route for the graft.

In some embodiments, the prediction means is configured to make predictions of changes in physiological parameters of the graft according to several scenarios of changes in operating setpoints of the transport case and to display these predictions.

In some embodiments, the device comprises a means for communicating with a central unit of a perfusion machine configured to receive physiological parameter values for the graft during the transport.

In some embodiments, the unit also comprises a control means configured to command one of two modes of operation of the unit as a function of the temperature measured, between:
a first mode of operation in which the unit has a nominal energy consumption, when the temperature of the graft is lower than a first predefined temperature threshold value; and
a second mode of operation in which the unit has a reduced energy consumption, when the temperature of the graft is higher than the first threshold value.

Thanks to these provisions, the risk that the unit itself participates in raising the temperature of the graft is reduced.

In some embodiments, the control means is configured to put the unit into sleep mode for a first predefined length of time if the temperature of the graft passes a second predefined temperature threshold value, higher than the first predefined temperature threshold value.

Thanks to these provisions, the graft is no longer heated by the unit.

In some embodiments, the unit also comprises a means for detecting the absence of movement, and a control means configured to command a third mode of operation of the unit, in which the unit has a reduced nominal consumption, when the unit is motionless during a length of time greater than a second predefined length of time, and/or to transmit an alert remotely when the unit is motionless during a length of time greater than a third predefined length of time.

Thanks to these provisions, when the unit is motionless, its autonomy is increased by reducing its electrical consumption and/or the people in charge of the transplant are warned of the graft's lack of movement, which can carry a risk for the success of this transplant.

In some embodiments, the unit also comprises a means for measuring the distance between the geographic position of the unit and at least one first predefined geographic position, and a control means configured to command one of two modes of operation of the device as a function of each distance measured, between:
a third mode of operation in which the unit transmits its geographic position, when each distance is greater than a predefined distance threshold value; and
a fourth mode of operation in which the communication means is inhibited, when at least one distance measured is less than said predefined distance threshold value.

Thanks to these provisions, the unit eliminates the risks of electromagnetic interference with the electronic systems of airports and aircraft.

In some embodiments, the unit comprises a distance measurement means, configured to measure a distance to the arrival point, measuring the distance between the geographic position of the unit and a second predefined geographic position, the destination of the graft, and a control means configured to command the sending of a message to at least one predefined destination terminal when the distance to the arrival point is less than a fourth predefined threshold value.

Thanks to these provisions, the medical team in charge of the transplant into the body of the recipient is notified in advance of the imminent arrival of the graft. This increases the probability of the operation being successful.

In some embodiments, the unit also comprises a distance measurement means, configured to measure a distance to the arrival point, measuring the distance between the geographic position of the unit and a second predefined geographic position, the destination of the graft, a lock configured to prohibit the opening of the transport case, and a control means configured to authorise the opening of the lock when the distance to the arrival point is less than a third predefined distance threshold value.

Thanks to these provisions, the transport case is locked during almost the entire transport time.

In some embodiments, the unit also comprises a distance measurement means configured to measure a distance between the geographic position of the unit and a predefined route, and a control means configured to trigger an alert remotely if the distance to the route is greater than a fourth predefined distance threshold value.

Thanks to these provisions, the people in charge of the transplant are warned of the change in the route of the graft, which can carry a risk for the success of this transplant.

In some embodiments, the unit also comprises an oximeter configured to measure the oxygen available in the graft and/or the graft's consumption of oxygen, and a control means configured to trigger an alert remotely when the oxygen available becomes less than a seventh predefined value and/or when the graft's consumption of oxygen falls below a predefined oximetry threshold value.

Thanks to these provisions, the risks of the graft deteriorating due to lack of oxygen are anticipated and can be taken into account by the people in charge of the graft transfer.

In some embodiments, the unit also comprises a hygrometer configured to measure the humidity in the atmosphere of the transport bag, and a control means configured to trigger an alert remotely when the humidity becomes greater than a predefined humidity threshold value.

Thanks to these provisions, the opening of the transport bag can be detected and this event can be taken into account.

In some embodiments, the unit also comprises a luminosity sensor configured to measure the luminosity in the transport bag, and a control means configured to trigger an alert remotely when the luminosity becomes greater than a predefined luminosity threshold value.

Thanks to these provisions, the opening of the transport bag or transport case can be detected and this event can be taken into account.

In some embodiments, the unit also comprises an accelerometer, and a control means configured to trigger an alert remotely when the acceleration becomes greater than a predefined acceleration threshold value.

Thanks to these provisions, a shock to the graft can be detected and taken into account.

### BRIEF DESCRIPTION OF THE FIGURES

Other advantages, aims and particular features of the invention will become apparent from the non-limiting description that follows of at least one particular embodiment of the device and method that are the subjects of the invention, with reference to drawings included in an appendix, wherein:

FIG. 1 represents, schematically, a first particular embodiment of the device that is the subject of the invention and systems with which the device communicates;

FIG. 2 represents, schematically, an electronic circuit of a second particular embodiment of the device that is the subject of the invention;

FIG. 3 represents, schematically and in a perspective view from above, a closed unit of the device whose electronic circuit is illustrated in FIG. 2;

FIG. 4 represents, schematically, the constituent elements of the chain of information, through to the hospital services, of the second embodiment of the device that is the subject of the invention;

FIG. 5 represents, in the form of a logic diagram, a first series of steps of a first particular embodiment of the method that is the subject of the invention;

FIG. 6 represents, in the form of a logic diagram, the continuation of the first series of steps illustrated in FIG. 5;

FIG. 7 represents, schematically, the constituent elements of the chain of information, through to the hospital services, of a third embodiment of the device that is the subject of the invention;

FIG. 8 represents, in the form of a logic diagram, a second series of steps of a second particular embodiment of the method that is the subject of the invention;

FIG. 9 represents, in the form of a logic diagram, the continuation of the second series of steps illustrated in FIG. 8; and

FIG. 10 represents, in the form of a logical diagram, a third series of steps of an embodiment of the invention.

### DESCRIPTION OF THE EMBODIMENTS

The present description is given in a non-limiting way, in which each characteristic of an embodiment can be combined with any other characteristic of any other embodiment in an advantageous way.

Note that the figures are not to scale.

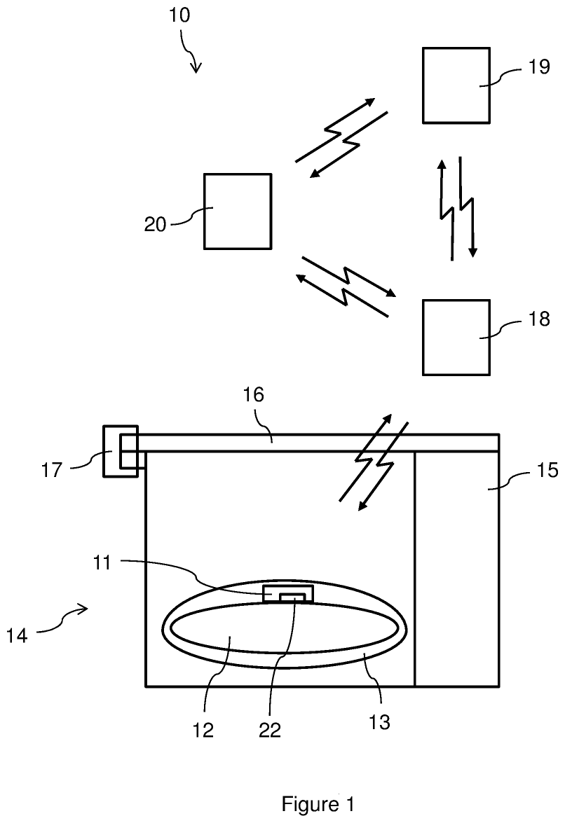

FIG. 1 represents, schematically, a system 10 for transporting a graft 12, the system 10 comprising a standalone unit 11 configured to accompany the graft 12 in a transport case 14 or in a transport bag 13 that is itself transported in the transport case 14. The standalone transport case 14, of a known type, comprises a unit 15 for maintaining environmental conditions around the bag 13 of the graft 12, and a cover 16. The term standalone here means that the unit 14 comprises or is connected to a mobile energy source allowing the graft to be transported. The transport case 14 is therefore preferably thermoregulated to maintain a temperature profile during the graft's transport, for example a fixed temperature (e.g. 10° C. or 37° C.). The unit 11 comprises a temperature sensor 22 that measures the temperature at the surface of the graft 12 or in the transport case 14.

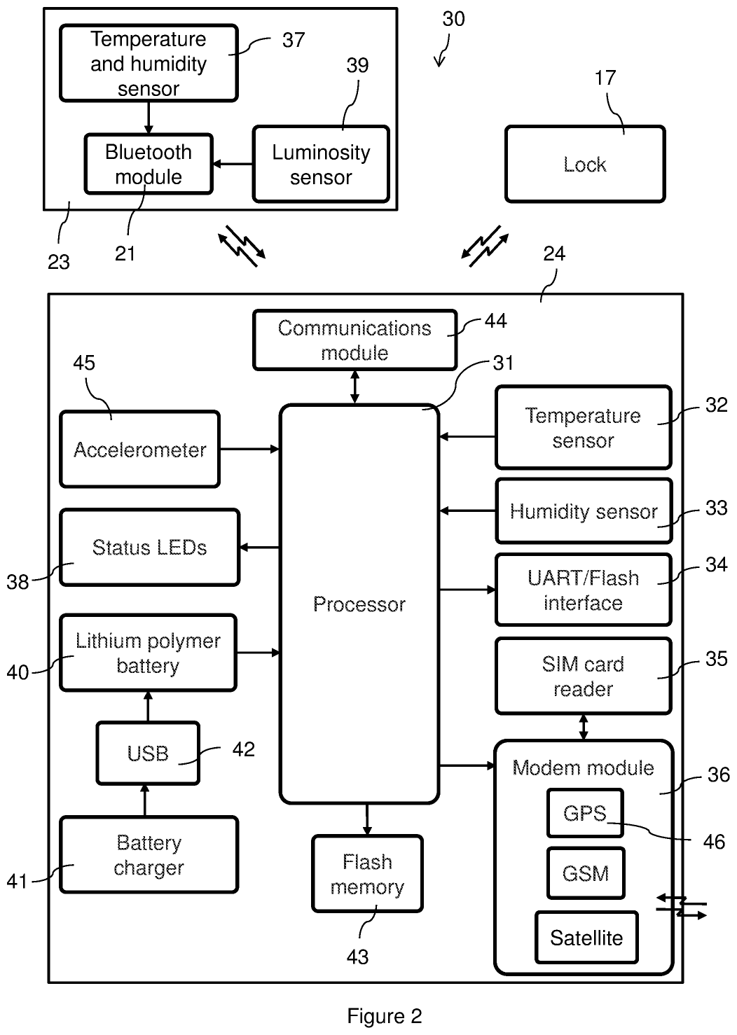

In the embodiment shown in FIG. 1, the unit 11 is in communication, for example via a cellular or satellite mobile telephony network 18, with a server 19 storing and processing the data measured, in particular temperature, and with at least one terminal 20 of the destination location of the graft, referred to as the “destination”. The unit 11 preferably also comprises a geographic position sensor (see FIG. 2), for example based on the processing of positioning signals from earth satellites. The unit 11 to be placed in the transport case 14 of a graft 12 is therefore preferably equipped with a geolocation means and also a telecommunications means to transmit the temperature and position values measured and help monitor the transport of the graft 12. Preferably, the unit 11 communicates with a lock 17 that blocks the opening of the transport case 14, to control the opening of the lock 17 and prevent an accidental opening of the case 14.

In the second embodiment, shown in FIGS. 2 to 4, a first standalone unit 23 is located inside the transport case and a second standalone unit 24 is located outside the transport case 14. In this way, the amount of heat generated inside the transport case is reduced while the remote communications range of the device is increased, this communication being performed by the unit 24. The units 23 and 24 communicate wirelessly with each other.

The electronic circuit 30 of the unit 24 comprises:
A control means comprising a processor 31;
A temperature sensor 32;
A humidity sensor 33, preferably positioned in the same location and incorporated into the same component as the temperature sensor 32;
A UART (for “Universal Asynchronous Receiver Transmitter”) flash interface 34. This interface is a communications port (communication means). It enables all the card's information to be accessed by connecting it with a computer;
A SIM (acronym for “Subscriber Identity/identification Module”) card reader 35, the SIM card being a chip containing a microcontroller and memory, used in mobile telephony to store information specific to the subscriber to a mobile network;
A modem module 36, preferably 3G/4G/5G/LTE, for example GSM or by satellite communications, and comprising a geolocation means 46 (“GPS”, for Global Positioning System). Preferably, the modem module 36 communicates with the microcontroller 31 via UART;
Indicator lights 38 indicating the status of the unit 24, for example light-emitting diodes;
A battery 40 recharged by a battery charger 41 linked to a connector 42 that is accessible from outside the unit 24, for example a USB (acronym for “Universal Serial Bus”, a serial bus standard) port. The electronic card is equipped with an electronic component making it possible to regulate the charge of the lithium battery 40. The 220V/5V charger 41 is external and is linked via the USB-C port 42;
A flash memory 43, a rewriteable solid-state mass memory, i.e. a memory having the characteristics of a random-access memory but whose data do not disappear when it is powered off;
A movement sensor 45, for example a MEMS (acronym for “Microelectromechanical systems”) type of accelerometer.

The circuit of the unit 23 comprises, connected to the microcontroller 31 by means of short-range communications components 21 and 44, for example a communications module utilising the Bluetooth (registered trademark) protocol:
A temperature and humidity probe 37 inside the transport case, i.e. for the temperature and humidity of the graft 12; and
A luminosity sensor 39 inside the transport case.

Optionally, an atmospheric pressure sensor (not shown) inside the case 14 is incorporated into the unit 23.

The processor 31, which implements a computer program stored in the flash memory 43, can therefore:
Communicate over a mobile telephone network, by means of the SIM card reader 35 and the modem module 36;
Know the geographic position of the unit 24, by means of the geolocation means 46 of the modem module 36;
Detect shocks, by means of the accelerometer 45;
Detect movements, by means of the geolocation means 46 of the modem module 36 and/or the accelerometer 45;
Measure the temperature of the graft, by means of the external temperature and humidity sensor 37;
Measure the humidity around the graft, by means of the humidity sensor 33;
Measure the luminosity inside the transport bag 13 of the graft 12, by means of the luminosity sensor 39; and
Indicate its operating status, by means of the indicator lights 38.

The processor 31 can therefore detect and report remotely the following events or incidents:
Rise in the temperature of the graft, for example because the transport case 14 is located in the sun or in a hot environment, or there is an air-conditioning failure, this rise triggering an immediate alert;
Shock to the graft, which could damage it;
Rise in the humidity and/or the level of light, indicating the graft bag being opened;
Altitude (determined by the pressure) and speed (determined by the geographic position) indicating periods of transport in an aircraft, i.e. in an airplane or helicopter;
Lack of movement of the transport case 14; and
Abnormal trajectory of the transport case 14 with regard to the expected destination.

Although the transport time of a graft is generally less than 24 hours, the battery 40 is sized for a longer operating time of the unit 24, for example several days or a week, with a communication period for the captured data of ten minutes, and the sensors read every second.

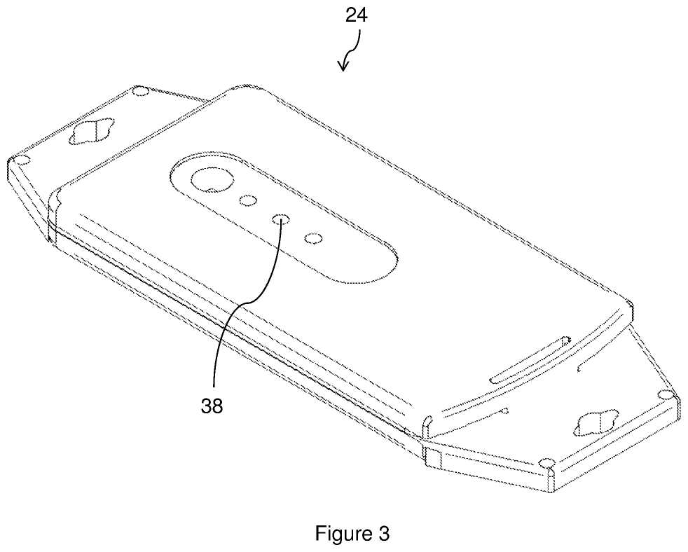

FIG. 3 shows a closed unit 24. Its dimensions are, for example, 9.5 centimetres×7 centimetres×3 centimetres.

Preferably, the unit 23, and, possibly, the unit 24 have an overall rectangular parallelepiped shape. The materials forming the outer surfaces of the unit 23 are of food grade and medical grade (USP, acronym for “US Pharmacopoeia”, class VI, which judges the suitability of plastic material intended for use as containers or accessories for parenteral preparations). These materials are chosen to prevent the diffusion of particles into the graft. These materials comprise, for example, ABS (acrylonitrile butadiene styrene, a thermoplastic polymer), PP (Polypropylene), PC (polycarbonate), PU (polyurethane), SEBS (polystyrene-b-poly(ethylene/propylene)-b-polystyrene). Preferably, the unit 23 has an overmoulding over the entire periphery of the unit and, in particular, on its corners. The overmoulding is made of a flexible material, for example flexible silicone, with rounded shapes, no protrusions, and a smooth surface. The units 23 and 24 are sealed (“IP68” leak-tightness, an index of protection against prolonged immersion).

The light-emitting diodes (“LED”) 38 are located on the upper surface of the unit 24. The two upper green LEDs make it possible to provide information about the battery level (the top LED=100−75%, and the LED below 75−25%). The bottom blue LED indicates the activity of the modem module 36 (for example, slow blinking means that the modem module 36 is switched on, and fast blinking means that the modem module 36 is connected to the network and/or sending data).

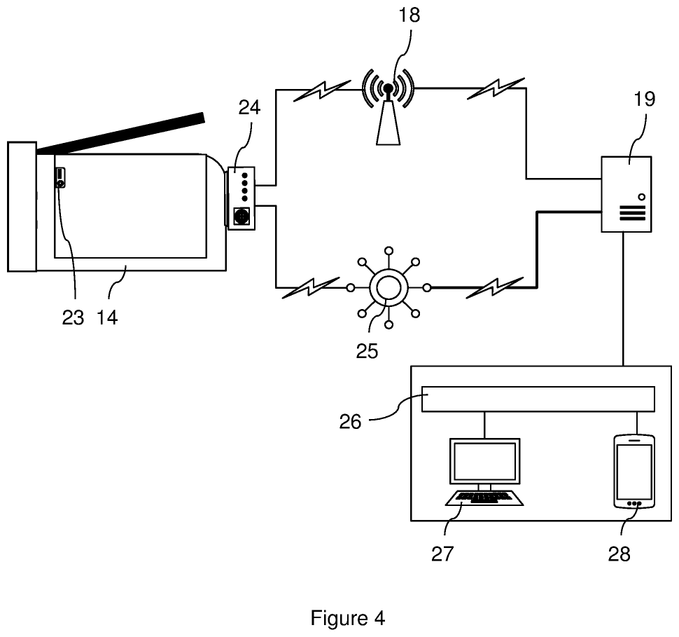

FIG. 4 shows constituent elements of the chain of information from the unit 24 through to the hospital services. FIG. 4 shows the units 23 and 24 associated to the transport case 14. The unit 24 communicates with the server 19 either via a mobile telephony network 18 or via a satellite communications network 25. The server stores the data it receives from the unit 24 and transmits information to computers 27 and communicating mobile terminals 28 of a computer network 26 of a hospital that is the destination of the graft 12.

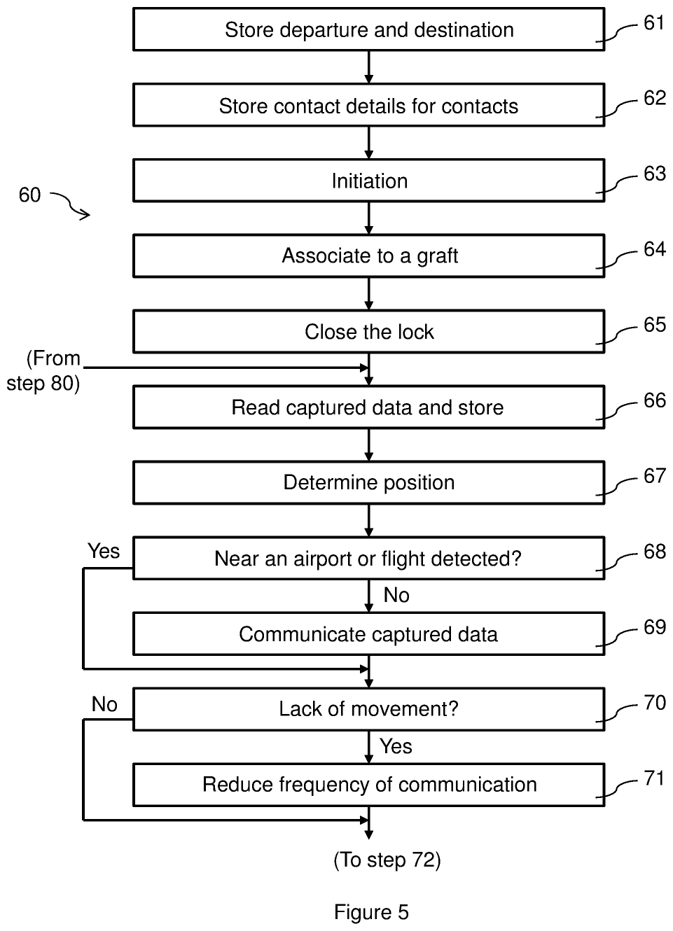

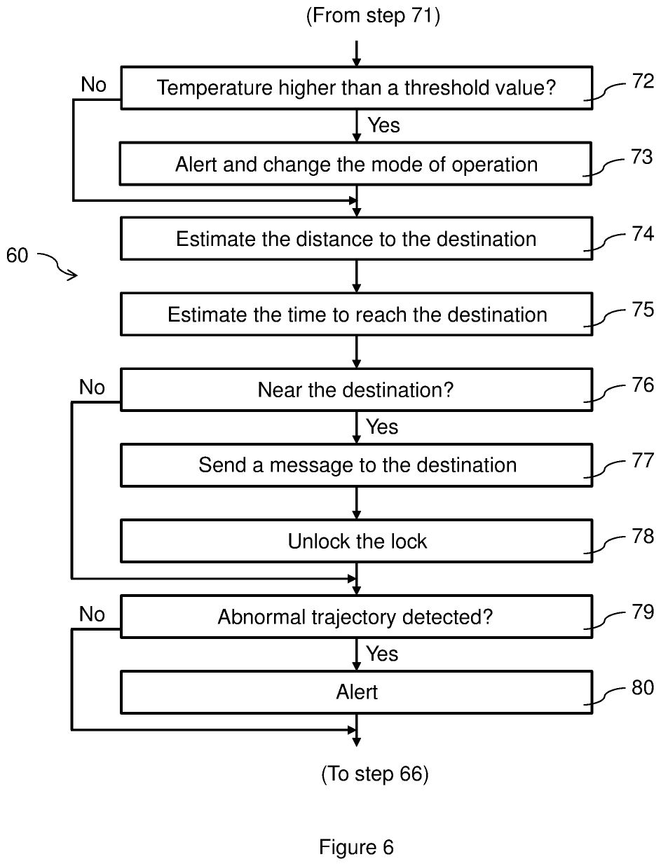

FIGS. 5 and 6 show steps in a particular embodiment 60 of the method that is the subject of the invention. This method 60 is intended to be implemented by the unit 24, possibly in conjunction with the server 19, and the unit 24 is designed to implement this method 60.

During a step 61, an operator loads into the memory of the unit the geographic position of the destination of the graft as the second predefined geographic position and, possibly, the position of the departure point and the planned route.

During a step 62, an operator or the server loads into the memory of the unit contact details for contacts, e.g. mobile telephone numbers and/or email addresses. For example, these are the contact details for the transplant coordinators, the recovery coordinators, the surgeons, the members of the transport team in charge of escorting the graft and/or the members of the medical team waiting for the graft to transplant it.

During a step 63, an operator initiates the regular operation of the unit. During a step 64, an operator physically associates the unit to a graft, by placing the unit in contact with the graft in a transport bag and this transport bag in a transport case that is itself put into operation to provide a suitable environment for the graft.

During a step 65, an operator closes the lock blocking the opening of the transport case. During a step 66, the unit collects the values captured for the temperature of the graft, external temperature, humidity, luminosity, geographic position and movement, and stores them in its memory.

During a step 67, the unit determines its geographic position. During a step 68, the unit or the server determines whether this geographic position is near an airport, i.e. at a distance less than a predefined distance threshold value from a first predefined geographic position, for example one kilometre, or whether the pressure and/or speed values represent a phase of transport in an airplane or helicopter for the transport case. If both tests give a negative result then, during a step 69, the unit transfers the data captured to the server 19 and proceeds to step 70. If the result of at least one of the tests in step 68 is positive, step 69 is not performed. In this way, the unit avoids generating electromagnetic disturbances for the electronic systems of the airport or aircraft.

Therefore, the processor 31 or the server 19 constitutes a means for measuring the distance between the geographic position of the unit and at least one first predefined geographic position, and a control means configured to command one of two modes of operation of the device as a function of each distance measured, between a first mode of operation, in which the device transmits its geographic position regularly, when each distance is greater than a first predefined distance threshold value, and a second mode of operation, in which the communication means is inhibited, when at least one distance measured is less than said first distance threshold value.

During the step 70, the unit determines whether it is motionless. If yes then, during a step 71, the processor 31 reduces the frequency with which data is communicated to the server 19, for example going from a 10-minute period to a 20-minute period. In this way, it increases its autonomy time and proceeds to step 72. If the result of the test 70 is negative, the processor 31 returns to the nominal frequency of communication and, during step 72, the unit determines whether the temperature of the graft is higher than a first temperature threshold value. For example, for a liver graft, which must remain between 2° C. and 6° C., the first threshold value can be 5.5° C. or 6° C.

If the result of the test 72 is positive then, during a step 73, the unit immediately sends an alert to all the contacts that it has contact details for, and changes the mode of operation to reduce its consumption of electricity and its generation of heat. For example, the unit switches from a nominal mode of operation to a degraded mode of operation that consumes less electrical energy. For example, the degraded mode of operation reduces the frequency of the measurements, and reduces or halts the local communications with the lock or sensors outside the unit, the determination of the geographic position, and displaying the displays.

Therefore the processor uses one of the two modes of operation of the device as a function of the temperature measured, between a first mode of operation in which the device has a nominal energy consumption, when the temperature of the graft is lower than a predefined temperature threshold value, and a second mode of operation in which the device has a reduced energy consumption, when the temperature of the graft is higher than this threshold value. However, the unit does transmit to the contacts and the server 19, at a faster frequency, for example every minute, the value captured for the temperature of the graft. The reverse change in the mode of operation is carried out as soon as the temperature of the graft falls below the first predefined threshold value.

In some embodiments, if the temperature of the graft passes another temperature threshold value, for example one degree Celsius higher than the first, the unit sends a new alert and goes into sleep mode for a predefined length of time, for example ten minutes, to temporarily halt any consumption of electricity likely to participate in heating the graft.

During a step 74, the unit determines its distance to the destination of the graft. During a step 75, the unit estimates the time needed to travel the remaining distance and arrive at the destination. During a step 76, the unit determines whether this distance to the destination and/or this time are lower than the distance (e.g. 20 kilometres) or time (e.g. 30 minutes) threshold values. If not, the unit proceeds to step 79. If yes then, during a step 77, the unit sends a message giving the contacts for the destination of the graft advance warning of the arrival of the graft. Then, during a step 78, if the distance to the destination is less than a distance threshold value, e.g. two kilometres (to take into account the extent of the largest hospital sites, whose position might have been simplified to a single point), the unit unlocks the lock 17.

During a step 79, the unit determines whether its position indicates an abnormal trajectory, for example being outside an ellipse comprising the departure and arrival points and their environment over ten or twenty kilometres and whose small radius is half the distance between the departure point and the arrival point. If yes then, during a step 80, the unit sends an alert message and its geographic position to all the contacts that it has contact details for. If not, the unit returns to step 66.

Note that the operation of the unit is determined by the destination team for the graft or by the server 19.

In all the steps described above in which the processor 31 makes a determination, in a variant it is the server 19 that makes this determination, alone or jointly with the unit.

In addition, the server 19 carries out a machine learning process, using feedback from medical teams (profile of the recipient, necrosis observed on the graft upon its arrival, graft rejection, disease in the transplanted organ, etc.), the best transport conditions for the graft (length of time, changes in the values captured, profile of the donor, etc.), risks incurred, by recipient profile, with a graft that reaches its destination.

The utilisation of the invention makes it possible to improve the logistics for all types of organ transplants. A transplant begins with the transplant surgeon's decision to initiate an action. This covers the recovery of the graft from the dead donor, its transport from the donor's establishment to the recipient's establishment, through to the transplant. With regard to a recovery coordinator utilising the device that is the subject of the invention, the successive steps are:
the recovery coordinator connects to a dedicated site hosted by the server 19, logs in and declares a new transport;
the coordinator enters the cristal number (anonymous identification number) of the donor, and the transplant type(s). He also chooses the recipient establishment and the transplant coordinator(s) of the establishment selected (he can have “all” the coordinators by default);
once he has validated the transport, to officially initiate and create the journey, the transplant coordinators of the selected establishment receive an alert via a notification, short message (“SMS”) or a call to their telephone;
the coordinator can then monitor the journey and the associated events directly.

When the transporter that he has chosen arrives, the recovery or transplant coordinator then physically associates the unit to the graft, in the transport bag, and places this bag in the transport case. Possibly, a visible code on the unit, for example a QR (acronym for “Quick Response”) code, is scanned to identify this unit.

Each contact can monitor the transplant's key steps and know the essential information thanks to the sensor, especially temperature, and verify that this is well within the required temperature range. If not, they will receive an alert notification.

It is also possible to know and validate the number of conservation bags that are used during the transplant. The bags of liquid for conserving the organs are used in order to increase the viability of the graft during the transport and for the patient waiting for the transplant. They are only used after the organ has been recovered. The organ is immersed in this conservation liquid, for example type IGL-1 or Custodiol (registered trademarks).

At the end of the journey, the coordinators can export the data to have better electronic management of their transplants.

As can be seen by reading the description above, the graft monitoring device that is the subject of the invention comprises, in a unit configured to accompany the graft in a transport bag which is itself transported in a transport case, a graft temperature sensor and a means for remotely communicating the temperature of the graft. Therefore, the unit is physically associated to the graft. It can monitor all the variations in transport conditions and, in particular, changes in the temperature of the graft. These transport conditions are regularly sent to the recovery and transplant coordinators, the surgeons, the members of the transport team in charge of escorting the graft and/or the members of the medical team waiting for the graft to transplant it.

In some embodiments, the unit also comprises a control means configured to command one of two modes of operation of the unit as a function of the temperature measured, between:
a first mode of operation in which the unit has a nominal energy consumption, when the temperature of the graft is lower than a first predefined temperature threshold value; and
a second mode of operation in which the unit has a reduced energy consumption, when the temperature of the graft is higher than the first threshold value.

In this way, the risk that the unit itself participates in raising the temperature of the graft is reduced.

In some embodiments, the control means is configured to put the unit into sleep mode for a first predefined length of time if the temperature of the graft passes a second predefined temperature threshold value, higher than the first predefined temperature threshold value. Thus, should the graft be close to a temperature of irreversible deterioration, it will no longer be heated by the unit.

In some embodiments, the unit also comprises a means for detecting the absence of movement, and a control means configured to command a third mode of operation of the unit, in which the unit has a reduced nominal consumption, when the unit is motionless during a length of time greater than a second predefined length of time, and/or to transmit an alert remotely when the unit is motionless during a length of time greater than a third predefined length of time. Therefore, when the unit is motionless, its autonomy is increased by reducing its electrical consumption and/or the people in charge of the transplant are warned of the graft's lack of movement, which can carry a risk for the success of this transplant.

In some embodiments, the unit also comprises a means for measuring the distance between the geographic position of the unit and at least one first predefined geographic position, and a control means configured to command one of two modes of operation of the device as a function of each distance measured, between:
a third mode of operation in which the unit transmits its geographic position, when each distance is greater than a predefined distance threshold value; and
a fourth mode of operation in which the communication means is inhibited, when at least one distance measured is less than said predefined distance threshold value.

As a result, the unit eliminates the risks of electromagnetic interference with the electronic systems of airports and aircraft.

In some embodiments, the unit comprises a distance measurement means, configured to measure a distance to the arrival point, measuring the distance between the geographic position of the unit and a second predefined geographic position, the destination of the graft, and a control means configured to command the sending of a message to at least one predefined destination terminal when the distance to the arrival point is less than a fourth predefined threshold value. Thus, the medical team in charge of the transplant into the body of the recipient is notified in advance of the imminent arrival of the graft. This increases the probability of the operation being successful.

In some embodiments, the unit also comprises a distance measurement means, configured to measure a distance to the arrival point, measuring the distance between the geographic position of the unit and a second predefined geographic position, the destination of the graft, a lock configured to prohibit the opening of the transport case, and a control means configured to authorise the opening of the lock when the distance to the arrival point is less than a third predefined distance threshold value. Thus, the transport case is locked during almost the entire transport time.

In some embodiments, the unit also comprises a distance measurement means configured to measure a distance between the geographic position of the unit and a predefined route, and a control means configured to trigger an alert remotely if the distance to the route is greater than a fourth predefined distance threshold value. Therefore, the people in charge of the transplant are warned of the change in the route of the graft, which can carry a risk for the success of this transplant.

In some embodiments, the unit also comprises an oximeter configured to measure the oxygen available in the graft and/or the graft's consumption of oxygen, and a control means configured to trigger an alert remotely when the oxygen available becomes less than a seventh predefined value and/or when the graft's consumption of oxygen falls below a predefined oximetry threshold value. The risks of the graft deteriorating due to lack of oxygen are therefore anticipated and can be taken into account by the people in charge of the graft transfer.

In some embodiments, the unit also comprises a hygrometer configured to measure the humidity in the atmosphere of the transport bag, and a control means configured to trigger an alert remotely when the humidity becomes greater than a predefined humidity threshold value. Thus, the opening of the transport bag can be detected and this event can be taken into account.

In some embodiments, the unit also comprises a luminosity sensor configured to measure the luminosity in the transport bag, and a control means configured to trigger an alert remotely when the luminosity becomes greater than a predefined luminosity threshold value. Thus, the opening of the transport bag or transport case can be detected and this event can be taken into account.

In some embodiments, the unit also comprises an accelerometer, and a control means configured to trigger an alert remotely when the acceleration becomes greater than a predefined acceleration threshold value. Therefore, a shock to the graft can be detected and taken into account.

A list is given below of operating parameters for all or part of the embodiments of the invention and determination parameters for threshold values, whose breach is monitored.
a/ first predefined temperature threshold value: set based on the graft and its sensitivity to the ambient temperature for the expected transport time. The value is determined by applying a safety margin with regard to the results of published scientific studies and/or by machine learning based on the graft rejection rate and/or other perturbations following the transplant (length of hospitalisation, return to hospital, postoperative medical treatments, feelings experienced by the recipient, etc.).
b/ second predefined temperature threshold value: set in the same way as the first predefined temperature threshold value, it has a lower safety margin than the first predefined temperature threshold value.
c/ first predefined length of time: the estimated time for cooling the graft to bring it below the first predefined temperature threshold value. It is preferably set based on the type of graft and the type of transport case.
d/ second predefined length of time: set based on normal stoppages (determined arbitrarily or by learning) for a graft transport (pause for leaving the parking area, at traffic lights, stop signs, tolls, breaks at motorway rest areas, etc.). The third predefined length of time is greater than the second predefined length of time.
e/ first predefined geographic position: this is the position of airports.
f/ first predefined distance threshold value: this is a perimeter representative of the airport area, for example a radius of two kilometres.
g/ second predefined geographic position: this is the position of the destination of the transport vehicle.
h/ second predefined distance threshold value: this is a distance corresponding to an advance notification required so the graft recipient's medical team can be in place. For example, this distance corresponds to a length of time, estimated by the vehicle's guide system, for the vehicle to travel to its destination, equal to the required notification time required, for example 30 minutes.
i/ predefined destination terminal: this consists of at least one telecommunications terminal of the graft recipient's medical team.
j/ third predefined distance threshold value: this is a perimeter around the destination location, for example corresponding to the grounds of a hospital where the graft recipient's medical team is located.
k/ predefined route: this is the route planned for the transport of the graft, for example estimated by the vehicle's guide system, to one airport, then from another airport, and the destination of the graft. This route can be re-evaluated during the transport, especially to take traffic conditions into account.
l/ fourth predefined distance threshold value: this is a tolerance around the predefined route, for example ten or twenty kilometres.
m/ predefined oximetry threshold value: set based on the graft and its sensitivity to its consumption of oxygen for the expected transport time. The value is determined by applying a safety margin with regard to the results of published scientific studies and/or by machine learning based on the graft rejection rate and/or other perturbations following the transplant (length of hospitalisation, return to hospital, postoperative medical treatments, feelings experienced by the recipient, etc.).
n/ predefined humidity threshold value: this is the humidity limit making it possible to detect the opening of the transport case.
o/ predefined luminosity threshold value: set to detect an opening of the transport case or transport bag, and not detect the light intensity emitted by the unit's light-emitting diodes.
p/ predefined acceleration threshold value: set based on the graft and its sensitivity to shocks. The value is determined by applying a safety margin with regard to the results of published scientific studies and/or by machine learning based on the graft rejection rate and/or other perturbations following the transplant (length of hospitalisation, return to hospital, postoperative medical treatments, feelings experienced by the recipient, etc.).

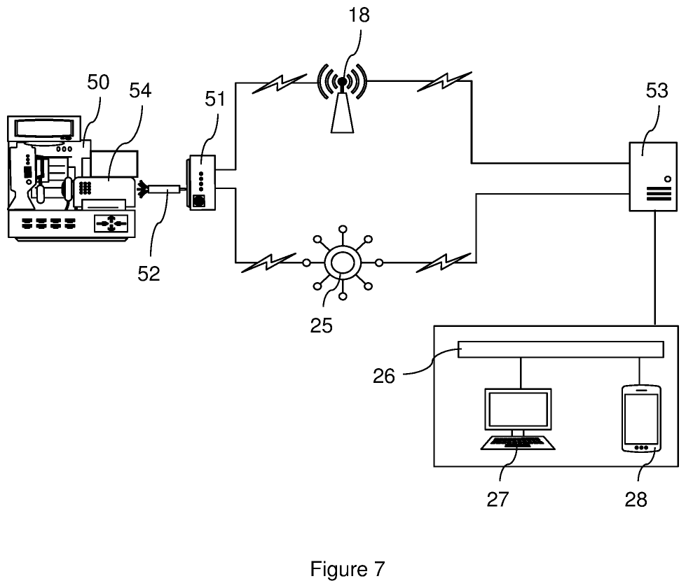

In the third embodiment, shown in FIG. 7 with the information transmission chain already shown in FIG. 4, the device comprises two units: one unit 51, similar to unit 24 except that it is connected to a mobile perfusion machine 50 comprising the transport unit 54. The connection between the perfusion machine 50 and the unit 51 is wired or wireless, and makes it possible for the unit 51 to collect the operating data and the values captured by the perfusion machine 50. In the embodiment shown in FIG. 7, the connection between the unit 51 and the perfusion machine 50 comprises USB connectors 52.

The central unit of the perfusion machine 50 can therefore supply the following physiological data, according to the graft considered:
Temperatures of the perfused blood, a graft preservation solution, an ice bag and/or a bubble trap;
Estimated level of glucose in the blood or in the graft;
Bile produced;
Potential of hydrogen, pH value;
Lactate level;
Level of enzymes;
Flow rates (cava vein, portal vein and/or artery);
Blood pressures (arterial, venous, systolic and diastolic pressures, and/or their mean over a predefined length of time);
Vascular resistance indices of the graft;
Level of gas (oxygen and/or air) in the blood;
Arterial blood gas;
Heart rate;
Haematocrit;
Mixed oxygen saturation;
Respiratory rate;
Tidal volume; and/or
Positive expiratory pressure.

The central unit of the perfusion machine 50 can also supply the following operating data for the machine:
Speed and/or flow rates of the pumps;
Operating error codes;
Graft identification number;
Length of perfusion;
Current date and time;
Open or closed status of the cover of the transport case; and/or
Charge level of the battery.

The server 53, which receives information from the sensors of the unit 51, and data and measurements from the perfusion machine 50, stores these values and processes them to make a prediction about changes in at least some of these values and assist the decision-making of the people in charge of the transport and transplant of the graft 12.

In a preferred variant (not shown), a unit 23 is associated to the graft in the transport case 54 of the perfusion machine 50. The unit 51 therefore receives data from the central unit of the perfusion machine 50 and from the unit 23. This variant has, in particular, the advantage of providing temperature measurement redundancy for the graft, which improves the reliability of the device.

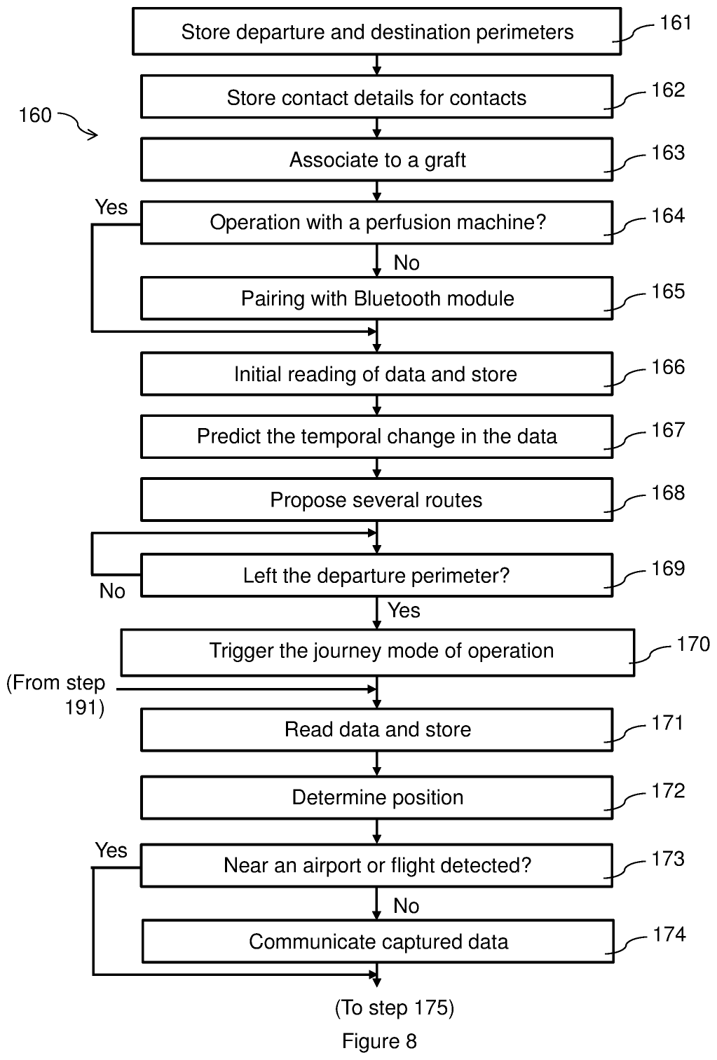

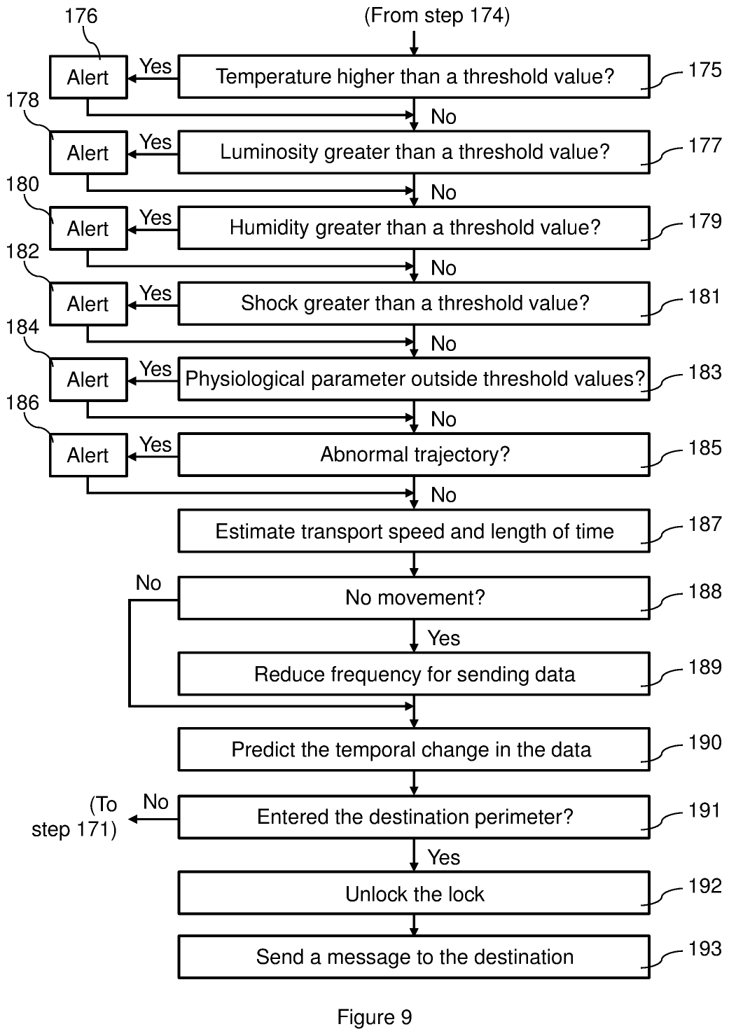

FIGS. 8 and 9 show an example of a series of steps 160 carried out jointly by the unit 51 and the server 53 during a graft transport. During a step 161, the geographic perimeters for the departure and destination of the graft are stored. These are, for example, the geographic coordinates of hospitals, with a radius of proximity, e.g. of ten kilometres. During a step 162, contact details of contacts (email addresses and telephone numbers) for the people in charge of the transport and transplant are stored. During a step 163, the graft 12 is placed in the transport case 14 or 54. The unit 51 therefore receives data from the central unit of the perfusion machine 50 and/or from the unit 23 placed in the transport case 14 or 54. In the case where the case is incorporated into or associated to a perfusion machine 50, the unit 51 and the central unit of this perfusion machine are connected, for example by means of USB connectors 52.

During a step 164, it is determined whether the transport case forms part of a perfusion machine, based on the presence of communication with this machine. If the result is negative then, during a step 165, the pairing of the units 23 and 24 is carried out. If the result is positive, or following the step 165, during a step 166 the unit 51 carries out an initial reading of the geographic position and physiological data captured by the sensors of the device and, possibly, by the sensors of the perfusion machine (see list above). The operating data (see list above) for the perfusion machine 50 can also be read by the unit 51 during this step.

During a step 167, a prediction is made of the temporal change in the values of the data captured or received. To this end, machine learning is utilised based on values observed during previous similar graft transports. This learning is preferably carried out by the server 53. This predicted change represents the graft's ability to be transplanted in the destination hospital, based on the transport time.

During a step 168, based on the data captured and predicted changes, one or more routes for the graft are proposed, for example by road, plain, train, etc. Possibly, this step takes into account traffic conditions, train or regular airline schedules, or the availability of private flights. A person in charge of the transport then selects a route for the graft.

During a step 169, it is determined whether the graft has left the departure perimeter, based on its geographic position. If not, this step 169 is repeated. Otherwise, if the graft has left the departure perimeter, during a step 170 the journey mode of operation is triggered, i.e. the transport itself. During a step 171, a new reading is carried out of the physiological data, geographic position and, possibly, operating data of the perfusion machine. During a step 172, a determination of the geographic position is carried out. During a step 173, it is determined whether the graft is near an airport. If not, during a step 174, the data captured are communicated to the server 19 or 53. If the graft is near an airport, or following the step 174, during a step 175, it is determined whether the temperature of the graft is higher than a threshold value. If yes, during a step 176, a local alert is triggered intended for the person in charge of the transport who is accompanying the graft, to inform him of this event. Following one of steps 175 and 176, during a step 177 it is determined whether the luminosity inside the transport case is greater than a threshold value. If yes, during a step 178, a local alert similar to the alert 176 is triggered. Following one of steps 177 and 178, during a step 179 it is determined whether the level of humidity inside the transport case is greater than a threshold value. If yes, during a step 180, a local alert similar to the alert 176 is triggered. Following one of steps 179 and 180, during a step 181 it is determined whether the graft has received a shock with a severity greater than a threshold value. If yes, during a step 182, a local alert similar to the alert 176 is triggered. Following one of steps 181 and 182, during a step 183 it is determined whether one of the other physiological parameters of the graft is outside a range delimited by two threshold values. If yes, during a step 184, a local alert similar to the alert 176 is triggered. Following one of steps 183 and 184, during a step 185 it is determined whether the geographic trajectory of the graft is normal, i.e. on a route close to the route selected at the end of step 168. If no, a local alert similar to the alert 176 is triggered.

During a step 187, an estimate is made of the graft's speed and the time remaining until the graft arrives at its destination. During a step 188, it is determined whether the graft is motionless. If yes, the frequency for sending data to the server 19 or 53 is reduced, during a step 189. If no, or after the step 189, during a step 190 a new prediction is made of the temporal change in the physiological parameters of the graft, its condition on arrival at its destination and this graft's ability to be transplanted, and the person in charge of the graft transport and the destination hospital are informed if one of the predicted values is outside an allowed range of values.

During a step 191, it is determined whether the graft is entering the destination perimeter. If not, return to step 171. Otherwise, if the graft has entered the destination perimeter, during a step 192 the lock is unlocked and then, during a step 193, a message is sent to the destination hospital to inform it of the graft's estimated arrival time.

As can be seen by reading the description above, the utilisation of the invention makes it possible to predict changes in the condition of the graft based on data measured or received, and machine learning based on similar data collected during transports already carried out. These predictions assist the decision-making of the people in charge of the transport and transplant.

Other predictions can also be made for the purposes of decision-making. In particular, these decisions concern the setpoint values to be applied to the perfusion machine, the possible immediate intervention on the graft in the event of decannulation, the possibility of proceeding with the transplant procedure, the choice of the recipient patient, the decision to keep the patient in hospital for longer after the transplant.

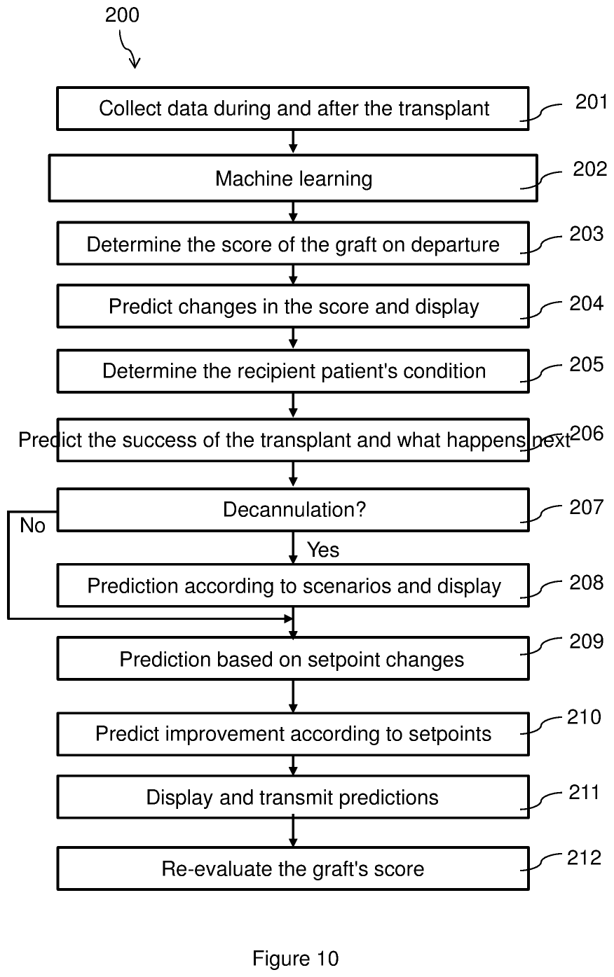

FIG. 10 shows steps that can be combined, fully or partially, with the steps described above with reference to FIGS. 5, 6, 8 and 9. During a step 201, data with dates are collected at the time of the transplant and/or after transplant into the patient's body. For example, the length of the transplant operation, the date the patient left the hospital, complications during the operations or in the following days, the date the patient returned to a hospital, the date of a new transplant, and the date of the patient's death can be stored.

During a step 202, the machine learning is carried out based on the transport data and the data collected during step 201. This provides a database of collected data and a system able to make predictions of changes in these data and the timescales and complications described above, according to data captured or read during a new graft transport and/or following the transplant.

During a step 203, a score is determined for the initial graft condition, prior to its transport. The evaluation of this score is taught in document PCT/FR2020/050437, incorporated herein by reference. This score is representative of the graft's ability to be implanted into the body of a recipient patient.

During a step 204, the progression of this score through to arrival at the destination is determined, according to the transport data (data collected or read, and times remaining until arrival at the destination, etc.) and based on machine learning.

During a step 205, the recipient patient's condition is determined, for example by entering his age, gender, weight, any pathologies, and his treatments while waiting for the transplant.

During a step 206, a prediction of the transplant's success and a prediction of the potential complications and timescales described above are made, based on machine learning.

This score and these predictions relating to the recipient patient can be transmitted to the destination hospital of the graft, to help it decide whether to prepare the patient for the transplant, or to abandon the transplant. In effect, a low initial low score, followed by transport with a transport time greater than the time initially anticipated, must lead to a decision on whether or not to proceed with the envisaged transplant and, possibly, to choose another graft recipient. Utilisation of the present invention makes it possible to document this decision with evaluations of the length of time the recipient will be kept in the hospital, the length of time before his subsequent return to a hospital, the need to perform another transplant, his life expectancy after the transplant, etc.

In the event of decannulation, detected during step 207, by the perfusion machine, the device that is the subject of the invention can display to this person in charge and transmit to the hospital, the predictions of changes in the score and/or physiological parameters through to arrival at the destination hospital according to different scenarios: the graft is kept in its current condition, therefore with no supply or with a partial supply, or this supply is restored. These predictions are made, displayed and transmitted during step 20. An informed decision can therefore be made about whether to:
Keep the graft with no blood or other inputs supplied; or
Open the transport case 54, and restore the supply to the graft, even if this means taking risks with regard to sterility during this intervention in the course of the transport.

Utilisation of the present invention also makes it possible to provide information to the person in charge of the transport with regard to a possible change in the transport conditions. For example, the level of glucose and its predicted changes are determined and displayed to this person in charge and transmitted to the destination hospital during a step 209, for several setpoint scenarios. This person can then decide whether to modify the operating setpoints of the perfusion machine to raise or lower this glucose level.

Utilisation of the present invention also makes it possible, during transport and based on predictions of the physiological values on arrival at the hospital, to improve the condition of the graft, for example by defatting a liver graft, according to known techniques. For this purpose, steatosis can be measured during the recovery of the graft, for example by implementing the teachings of document PCT/FR2020/050437, and then evaluated and predicted for the arrival at the destination, based on several levels of operating setpoints for the perfusion machine 50. The person in charge of the transport or transplant can therefore choose the operating setpoints of this machine 50 to optimise the graft's condition on arrival at the destination.

During a step 210, the device makes a prediction of the improvement in the graft's score based on the setpoints applied to the perfusion machine. During a step 211, these predictions are displayed locally and transmitted to the destination hospital.

Optionally, a camera is positioned with a light source inside the case 54 to carry out successive evaluations of the graft's steatosis (or another parameter having visible consequences on the surface of the graft), during a step 212.

For all the steps displaying a prediction based on different scenarios of operating setpoint values for the perfusion machine described above, in a variant, the device selects one of these scenarios based on the result of at least one of these predictions and applies at least one setpoint value that it represents to the perfusion machine.

Lastly, machine learning makes it possible to investigate new relationships between the values of transport conditions or physiological parameters of the graft during its recovery or transport and the results of the transplant in terms of the timescales expressed above and the patient's quality of life.

Utilisation of the present invention also enables optimum use to be made of the data (parameters) retrieved by a perfusion machine (which can help the decision-making of the people in charge of the transport and transplant). Retrieving the date captured by the perfusion machine, a prediction is made of changes in these data. The device can therefore provide advice on the best route of the organ with regard to changes in the parameters and environmental conditions. The device can also recommend, based on data measured or predicted, the actions to be carried out during the transport.

1. A device for monitoring a graft which comprises, in a standalone unit configured to accompany the graft in a graft transport case, a graft temperature sensor and a means for remotely communicating the temperature of the graft.

2. The device according to claim 1, which also comprises sensors capturing values of physiological parameters of the graft.

3. The device according to claim 2, which also comprises a means for making predictions about changes in the values of physiological parameters while the graft is being transported.

4. The device according to claim 3, wherein the prediction means is based on machine learning performed using physiological parameter values obtained during previous transports.

5. The device according to claim 3, wherein the prediction means is configured to make at least one prediction of physiological parameter values for the time when the graft arrives at the site of its transplant into the body of a recipient patient.

6. The device according to claim 5, which comprises a means for geolocation and evaluation at the time when the graft arrives at the site of its transplant into the body of a recipient patient.

7. The device according to claim 3, wherein the prediction means is configured to propose at least one route for the graft.

8. The device according to claim 3, wherein the prediction means is configured to make predictions of changes in physiological parameters of the graft according to several scenarios of changes in operating setpoints of the transport case and to display these predictions.

9. The device according to claim 1, which comprises a means for communicating with a central unit of a perfusion machine configured to receive physiological parameter values for the graft during the transport.

10. The device according to claim 1, wherein the unit also comprises a control means configured to command one of two modes of operation of the unit as a function of the temperature measured, between:
a first mode of operation in which the unit has a nominal energy consumption, when the temperature of the graft is lower than a first predefined temperature threshold value; and
a second mode of operation in which the unit has a reduced energy consumption, when the temperature of the graft is higher than said first threshold value.

11. The device according to claim 10, wherein the control means is configured to put the unit into sleep mode for a first predefined length of time if the temperature of the graft passes a second predefined temperature threshold value, higher than the first predefined temperature threshold value.

12. The device according to claim 1, wherein the unit also comprises a means for detecting the absence of movement, and a control means configured to command a third mode of operation of the unit, in which the unit has a reduced nominal consumption, when the unit is motionless during a length of time greater than a second predefined length of time, and/or to transmit an alert remotely when the unit is motionless during a length of time greater than a third predefined length of time.

13. The device according to claim 1, wherein the unit also comprises a means for measuring the distance between the geographic position of the unit and at least one first predefined geographic position, and a control means configured to command one of two modes of operation of the device as a function of each distance measured, between:
a third mode of operation in which the unit transmits its geographic position, when each distance is greater than a first predefined distance threshold value; and
a fourth mode of operation in which the communication means is inhibited, when at least one distance measured is less than said first predefined distance threshold value.

14. The device according to claim 1, wherein the unit comprises a distance measurement means, configured to measure a distance to the arrival point, measuring the distance between the geographic position of the unit and a second predefined geographic position, the destination of the graft, and a control means configured to command the sending of a message to at least one predefined destination terminal when the distance to the arrival point is less than a second predefined distance threshold value.

15. The device according to claim 1, wherein the unit comprises a distance measurement means, configured to measure a distance to the arrival point, measuring the distance between the geographic position of the unit and a second predefined geographic position, the destination of the graft, a lock configured to prohibit the opening of the transport case, and a control means configured to authorise the opening of the lock when the distance to the arrival point is less than a third predefined distance threshold value.

16. The Device according to claim 1, wherein the unit also comprises a distance measurement means configured to measure a distance between the geographic position of the unit and a predefined route, and a control means configured to trigger an alert remotely if the distance to the route is greater than a fourth predefined distance threshold value.

17. The device according to claim 1, wherein the unit also comprises an oximeter configured to measure the oxygen available in the graft and/or the graft's consumption of oxygen, and a control means configured to trigger an alert remotely when the oxygen available becomes less than a seventh predefined value and/or when the graft's consumption of oxygen falls below a predefined oximetry threshold value.

18. The device according to claim 1, wherein the unit also comprises a hygrometer configured to measure the humidity in the atmosphere of the transport bag, and a control means configured to trigger an alert remotely when the humidity becomes greater than a predefined humidity threshold value.

19. The device according to claim 1, wherein the unit also comprises a luminosity sensor configured to measure the luminosity in the transport bag, and a control means configured to trigger an alert remotely when the luminosity becomes greater than a predefined luminosity threshold value.

20. The device according to claim 1, wherein the unit also comprises an accelerometer, and a control means configured to trigger an alert remotely when the acceleration becomes greater than a predefined acceleration threshold value.
